(19) United States
(12) Patent Application Publication
(10) Pub. No.: US 2025/0375230 A1
(43) Pub. Date: Dec. 11, 2025

(54) Bone Plate System

(71) Applicant: Stryker European Operations Limited, Carrigtwohill (IE)

(72) Inventors: Robert Dominik, Grandvaux (CH); Joêl Bouquet, Lommiswil (CH); Isabel Keil, Freiburg (DE); Tobias BIüchel, Selzach (CH); Pierre-Luc Sylvestre, Grenchen (CH)

(21) Appl. No.: 18/737,240

(22) Filed: Jun. 7, 2024

(51) Int. Cl. A61B 17/80; A61B 17/86

(52) U.S. Cl. CPC A61B 17/8061; A61B 17/86; A61B 17/8695

(57) ABSTRACT

A bone plate system is disclosed. The system includes a plurality of anatomically designed plates and a plurality of utility plates. The system also includes different instrument sets for use with the plates and a screw rack for housing different screw sizes in a discrete and easy to access fashion.

### BACKGROUND

Bone plates are commonly used for osteosynthesis applications, in particular for the treatment of bone fractures and other procedures requiring bone stabilization. A typical fracture fixation procedure involves fixing a single bone plate to bone portions on either side of a fracture. This can be accomplished through the use of various fixation devices utilized in conjunction with the plates, including locking and non-locking screws, wires or the like. A reduction of the fracture may also be performed, either utilizing secondary tools or through the use of compression holes formed in the bone plate. The latter may include the fixing of the bone plate on one side of the fracture and inserting a screw into a compression hole located on the opposite side, which is typically designed to cause a translation of the screw therein, thereby translating the underlying bone portion to reduce the fracture.

Bone plate systems are typically designed for use in connection with specific bones (i.e., anatomically designed). For instance, it is common to see systems with plates directed to a single use, such as in connection with the distal femur, proximal tibia or the like. This of course means that situations involving multiple bone fractures (e.g., in connection with car accidents and other large trauma events) require multiple systems to be utilized in a single surgery. Each system may bring with it its own instruments and/or anchoring elements, thereby overpopulating the operating room with equipment and components. This of course runs the risk of increasing the chances of over complicating the surgery.

Thus, there exists a need for a system that can be utilized in connection with multiple bones to treat a single patient requiring multiple orthopedic interventions.

### BRIEF SUMMARY

The present inventions address the aforementioned drawbacks by providing a single bone plate system that includes bone plates for use in connection with multiple bones and portions of bones. The system additionally includes common anchoring elements and tooling for use with the various bone plates. Thus, systems disclosed herein are essentially a “one stop shop” for treating patients with fractures or other issues in connection with multiple bones. In addition to the system as a whole, certain individual components thereof are also of import. Indeed, the system includes many different that components that address drawbacks with existing systems and/or components.

Among the components included in the system are instrument sets/trays that house various instruments for use in implanting one or more plates of the systems. These trays are designed for ease of use and may include color coding or the like to permit the easy identification of tools for a particular use or in connection with a particular component, such as specifically sized screws. The system may also include a screw rack that is ergonomically designed and organized. The rack preferably includes various size screws organized in a manner that is easily identified by a surgeon or other medical professional. The screws included in the rack are also preferably determined based upon the typical requirements for surgical procedures—i.e., the quantities of such screw are driven by data determined from analyzing typical procedures. The rack may also include measuring scales and slots or the like for holding washers or other secondary components.

An aspect of the present invention is a bone plate system including a plurality of humerus plates, a plurality of femur plates, a plurality of tibia plates and a plurality of fibula plates.

In accordance with other embodiments of this aspect of the present invention, the humerus plates may include a proximal humerus plate, a proximal humerus posterior plate and an extra articular distal humerus plate. The femur plates may include a distal lateral femur plate, a distal medial femur plate and a distal medial femur plate. The tibia plates may include a proximal lateral tibial plate, a partial articular proximal tibia plate, an extra articular proximal tibial plate, a proximal medial tibial plate and a proximal posteromedial tibial plate. The tibia plates may further include a distal anterolateral tibial plate, a distal medial tibia plate and a distal posterior tibia plate. The fibula plates may include a distal lateral fibula plate, a distal posterolateral fibula plate and a distal posterior fibula plate. The bone plate system may also further include a plurality of utility plates. The utility plates may include straight plates, t-shaped plates and hook plates.

The bone plate system may also include a tray of tooling for use with small fragment plates and screws. The bone plate system may also include a tray of tooling for use with large fragment plates and screws. The bone plate system may also include a screw rack including a plurality of differently sized screws. The screw rack may include a screw measuring scale and/or a plurality of washer slots. In the screw rack, screws may be arranged in discrete locations based upon size. The screw rack may also include a removable cover.

Another aspect includes the ornamental designs for the various plates and other components described in the present application.

### BRIEF DESCRIPTION OF THE DRAWINGS

The patent or patent application file contains at least one drawings executed in color. Copies of this patent or patent application publication with color drawing(s) will be provided by the Office upon request and payment of the necessary fee.

A more complete appreciation of the subject matter of the present inventions and of the various advantages thereof can be realized by reference to the following detailed description in which reference is made to the accompanying drawings in which:

FIG. 1 is an illustration of all types of plates included in a system according to one embodiment of the present inventions.

FIG. 2 is an illustration of a tray of tooling for use with small fragment plates and screws of the system of FIG. 1.

FIG. 3 is an illustration of a tray of tooling for use with large fragment plates and screws of the system of FIG. 1.

FIG. 4 is an illustration of a screw rack for use with the system of FIG. 1.

FIG. 5 is a perspective view of a proximal humerus plate of the system of FIG. 1 shown implanted on a proximal portion of a humerus.

FIGS. 6A-H are different views of the proximal humerus plate of FIG. 5.

FIG. 7 is a perspective view of a proximal humerus posterior plate of the system of FIG. 1 shown implanted on a proximal portion of a humerus.

FIGS. 8A-H are different views of the proximal humerus posterior plate of FIG. 7.

FIG. 9 is a perspective view of an extra articular distal humerus plate of the system of FIG. 1 shown implanted on a distal portion of a humerus.

FIGS. 10A-H are different views of the extra articular distal humerus plate of FIG. 9.

FIG. 11 is a perspective view of a distal lateral femur plate of the system of FIG. 1 shown implanted on a distal portion of a femur.

FIGS. 12A-H are different views of the distal lateral femur plate of FIG. 11.

FIG. 13 is a perspective view of a first distal medial femur plate of the system of FIG. 1 shown implanted on a distal portion of a femur.

FIGS. 14A-H are different views of the first distal medial femur plate of FIG. 13.

FIG. 15 is a perspective view of a second distal medial femur plate of the system of FIG. 1 shown implanted on a distal portion of a femur.

FIGS. 16A-H are different views of the second distal medial femur plate of FIG. 15.

FIG. 17 is a perspective view of a proximal lateral tibia plate of the system of FIG. 1 shown implanted on a proximal portion of a tibia.

FIGS. 18A-H are different views of the proximal lateral tibia plate of FIG. 17.

FIG. 19 is a perspective view of a partial articular proximal tibia plate of the system of FIG. 1 shown implanted on a proximal portion of a tibia.

FIGS. 20A-H are different views of the partial articular proximal tibia plate of FIG. 19.

FIG. 21 is a perspective view of an extra articular proximal tibia plate of the system of FIG. 1 shown implanted on a proximal portion of a tibia.

FIGS. 22A-H are different views of the extra articular proximal tibia plate of FIG. 21.

FIG. 23 is a perspective view of a proximal medial tibia plate of the system of FIG. 1 shown implanted on a proximal portion of a tibia.

FIGS. 24A-H are different views of the proximal medial tibia plate of FIG. 23.

FIG. 25 is a perspective view of a proximal posteromedial tibia plate of the system of FIG. 1 shown implanted on a proximal portion of a tibia.

FIGS. 26A-H are different views of the proximal posteromedial tibia plate of FIG. 25.

FIG. 27 is a perspective view of a distal anterolateral tibia plate of the system of FIG. 1 shown implanted on a distal portion of a tibia.

FIGS. 28A-H are different views of the distal anterolateral tibia plate of FIG. 27.

FIG. 29 is a perspective view of a distal medial tibia plate of the system of FIG. 1 shown implanted on a distal portion of a tibia.

FIGS. 30A-H are different views of the distal medial tibia plate of FIG. 29.

FIG. 31 is a perspective view of a distal posterior tibia plate of the system of FIG. 1 shown implanted on a distal portion of a tibia.

FIGS. 32A-H are different views of the distal posterior tibia plate of FIG. 31.

FIG. 33 is a perspective view of a distal lateral fibula plate of the system of FIG. 1 shown implanted on a distal portion of a fibula.

FIGS. 34A-H are different views of the distal lateral fibula plate of FIG. 33.

FIG. 35 is a perspective view of a distal posterolateral fibula plate of the system of FIG. 1 shown implanted on a distal portion of a fibula.

FIGS. 36A-H are different views of the distal posterolateral fibula plate of FIG. 35.

FIG. 37 is a perspective view of a distal posterior fibula plate of the system of FIG. 1 shown implanted on a distal portion of a fibula.

FIGS. 38A-H are different views of the distal posterior fibula plate of FIG. 37.

FIGS. 39A-G are views of different utility plates of the system of FIG. 1.

FIG. 40 is a top view of a hybrid locking/compression hole according to another aspect of the present inventions.

### DETAILED DESCRIPTION

In describing the preferred embodiments of the inventions, specific terminology will be used for the sake of clarity. However, the inventions are not intended to be limited to any specific terms used herein, and it is to be understood that each specific term includes all technical equivalents, which operate in a similar manner to accomplish a similar purpose. In the drawings and in the description which follows, the term “proximal” means closer to the heart and the term “distal” means more distant from the heart. The term “anterior” means towards the front part of the body or the face and the term “posterior” means towards the back of the body. The term “medial” means toward the midline of the body and the term “lateral” means away from the midline of the body.

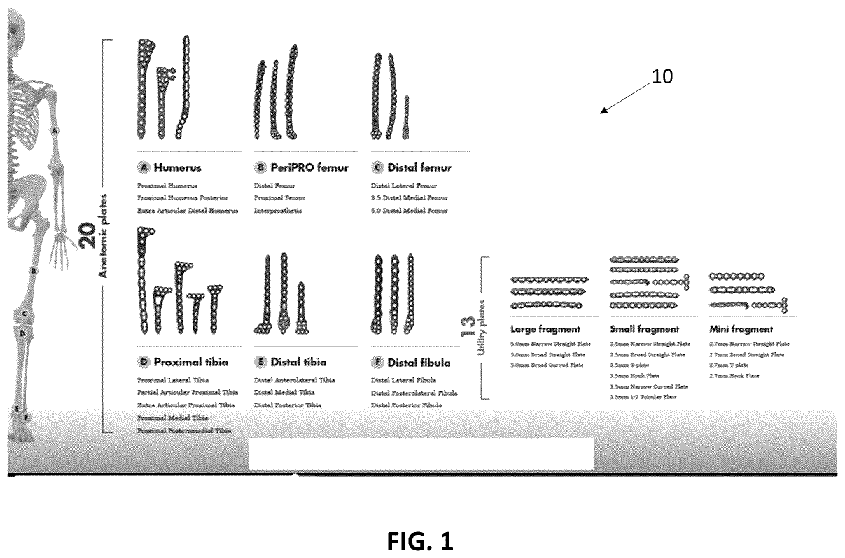

With reference to FIG. 1, a system 10 includes a plurality of plates for use in connection with various bones of the human body. As shown, system 10 includes plates anatomically designed for use in connection with humerus, femur, tibia and fibula bones, and in connection with different portions of those bones. System 10 also includes several utility plates that are useable in connection with certain of those bones and others within the body. The individual plates of system 10 will be discussed more fully below.

The anatomically specific plates of system 10 have been designed to be as anatomically accurate as possible. Such plates have also been designed in various sizes to be useable in connection with as many differently sized patients as possible. The particular designs shown have been designed using Stryker Orthopedics Modeling and Analytics (“SOMA®”) software. This resulted in evidence-based screw hole placement designed to offer a wide range of trajectory options for particular anatomy, as well as anatomical fit, creating contoured plates with left and right specific options. It is contemplated to further refine system 10 to include plates for a particular type of patient, for instance, male/female and ethnic-specific.

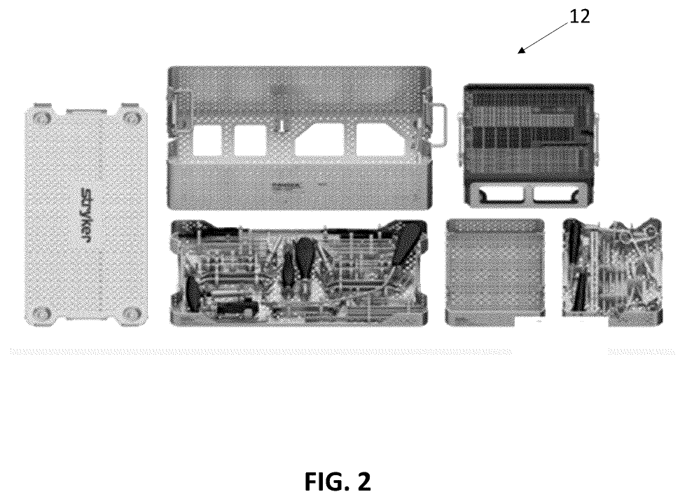

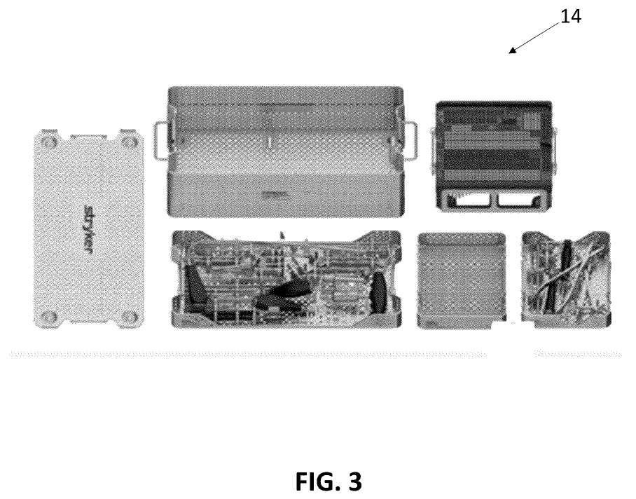

FIGS. 2 and 3 show instrument trays 12, 14 for use in connection with implanting the plates of system 10. In particular, tray 12 includes instruments for use with small fragment plates and screws, while tray 14 includes instruments for use with large fragment plates and screws. The various instruments included in each tray are color coded so as to dictate their particular use. For instance, instruments designed for use with 2.7 mm screws are colored purple, instruments designed for use with 3.5/4.0 mm screws are colored yellow, instruments designed for use with 4.0/4.5/6.0 mm screws are colored orange and instruments designed for use with 5.0 mm screws are colored blue. Of course, any color scheme can be employed in other embodiments. The color coding makes it easier to equate the correct tooling to a given screw situation. Coupled with the below discussed screw rack, system 10 provides for an ergonomically easy to use system by surgeons and other medical professionals.

Although not shown in detail, the instruments included in trays 12, 14 are of the type useful for implanting the various plates of system 10. This may include screw drivers, torque drivers, torque limiting handles, depth measuring instruments, depth gauges, screw capture sleeves, countersinks, taps, plate fixators, k-wires, reduction and retraction devices, forceps, hooks, retractors, plate benders, plate cutters, drill guides, other guides and the like. Of course, other instruments are contemplated, including powered devices such as powered drills and screw drivers. System 10 may also be designed to facilitate the use of a robot or other automated or semi-automated surgical device.

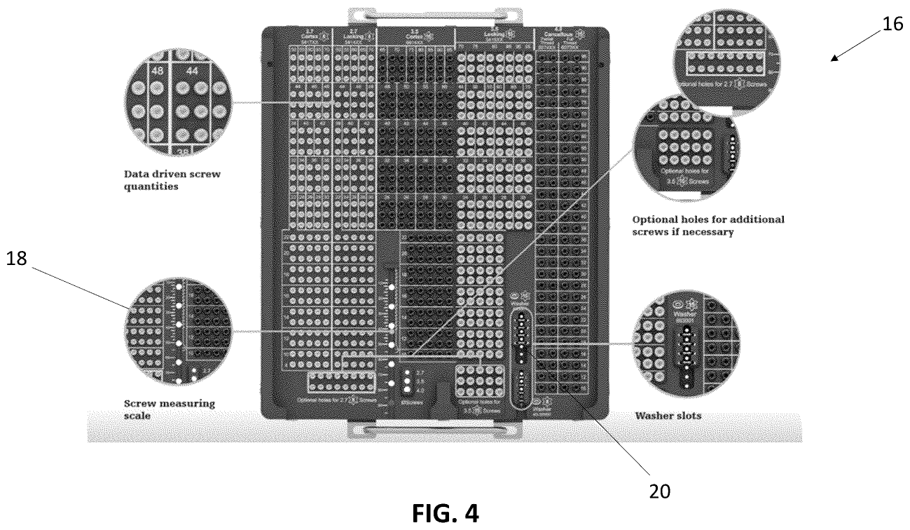

System 10 also includes a screw rack 16, shown in FIG. 4. Rack 16 provides for screw organization in a manner that makes screw selection as easy as possible. Not only are the quantities of screws included in rack 16 driven by data pertaining to typical procedures, but the screws are arranged in clear sections pertaining to size and/or use. This includes permitting the screws to be directly engaged by a driver and removed from rack 16 without the need for any intermediate intervention. Rack 16 also includes a screw measuring scale 18 to ensure the proper length/diameter screw is being utilized, as well as slots 20 for holding and dispensing washers for use during certain surgical procedures. It is contemplated that rack 16 may hold other components such as k-wires or the like. Rack 16 includes a cover (not shown) for proper sealing during transport and/or sterilization. Moreover, rack 16 is designed to be situated in a manner in which a surgeon or other medical professional can very easily obtain screws or other components therefrom.

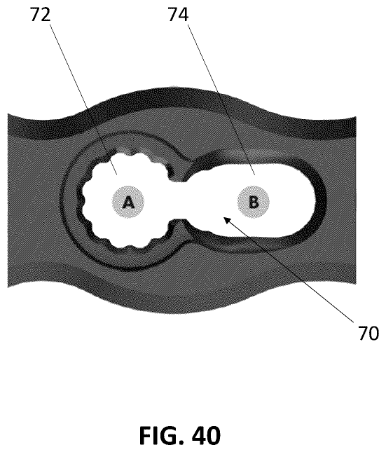

The various plates included in system 10 will now be discussed. Generally, each of the plates includes a variety of plate holes, including locking and non-locking screw holes. For instance, the various plates include variable angle locking holes in accordance with those disclosed in U.S. patent application Ser. No. 17/332,090 (“the '090 application”), now published as U.S. Patent Application Publication No. 2021/0282824, the disclosure of which is hereby incorporated by reference herein. While a specific embodiment screw hole is employed, it is also contemplated to employ any of the other plate hole designs disclosed in the '090 application in other embodiments. Certain of the plates also include other types of holes, such as threaded holes, elongate compression holes and hybrid locking/compression holes (see FIG. 40). Other screw holes designs known in the art can also be used.

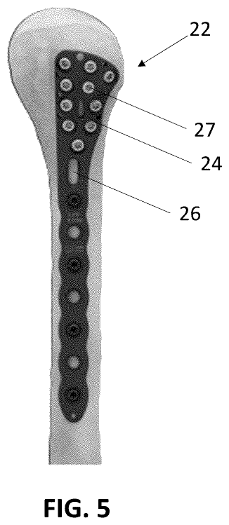

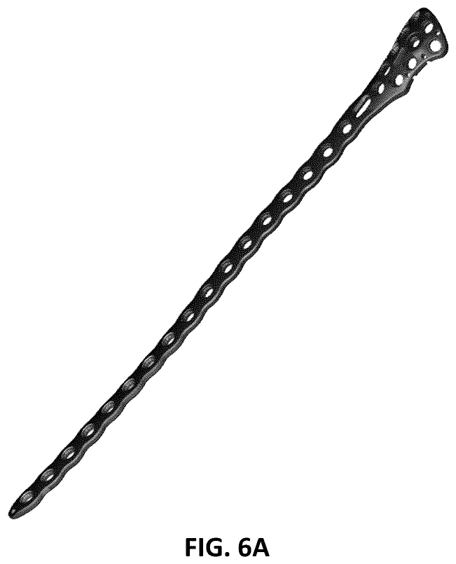

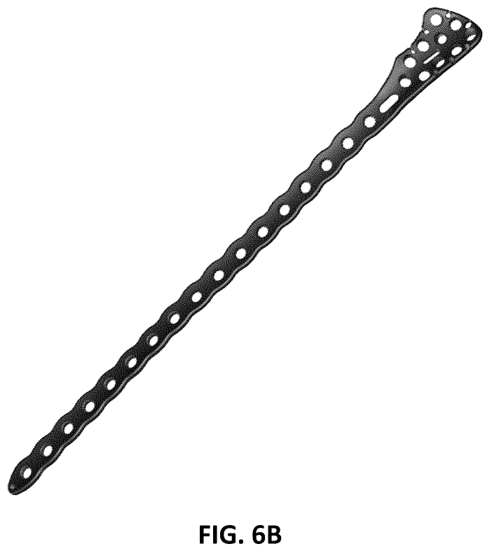

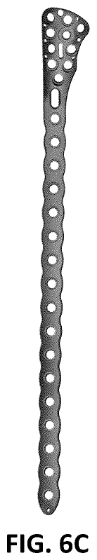

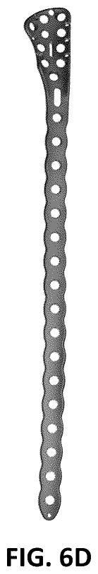

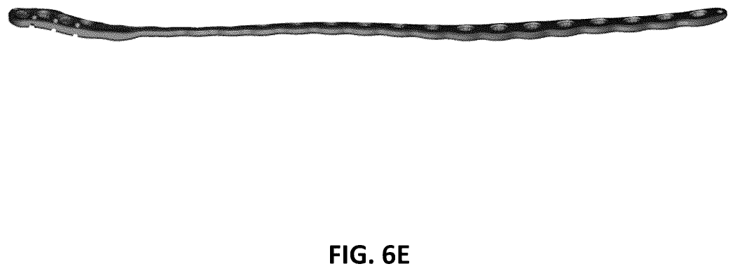

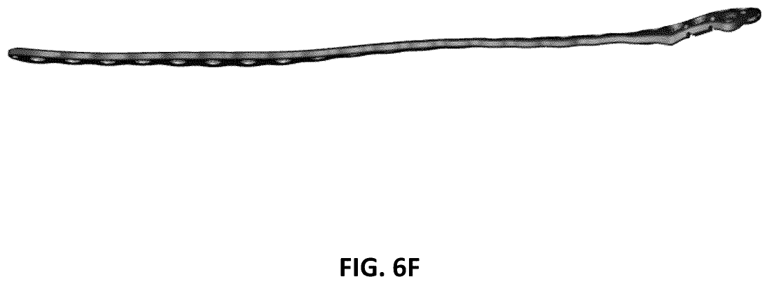

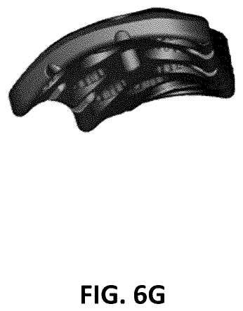

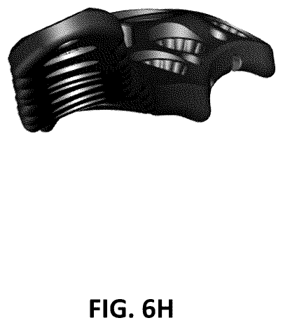

With reference to FIGS. 5 and 6A-H, a proximal humerus plate 22 is shown. In addition to various variable angle locking holes, plate 22 includes two slots 24, 26 that are useful in reducing fractures during a surgical procedure. Slot 24 is designed to accept a k-wire, while slot 26 is designed to accept a compression screw. Moreover, plate 22 is designed for the particular placement on the proximal humerus as shown in FIG. 5 such that a head portion generally aligns with the head of the bone and a shaft generally aligns with the shaft of the bone. Each portion of the plate includes a plurality of holes that are arranged in a manner that allows for typical fractures to be treated with the plate. Plate 22 also includes a threaded monoaxial hole 27, which can accept a locking screw as well as a threaded guide post (not shown) for connecting other components, like drill guides. Plate 22, like other plates in system 10, includes rounded and tapered ends designed for insertion under soft tissue and may employ a hole layout that permits converging and diverging screw trajectories.

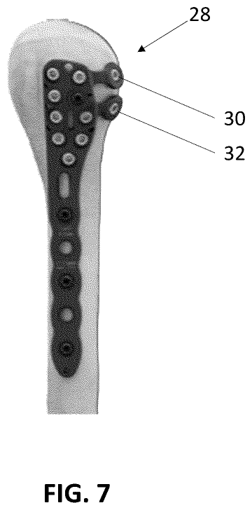

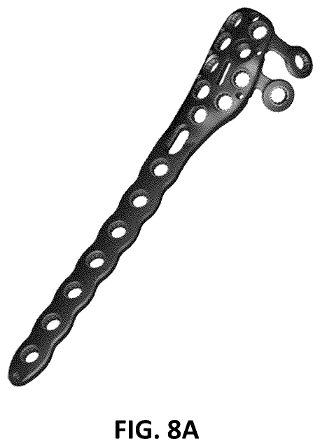

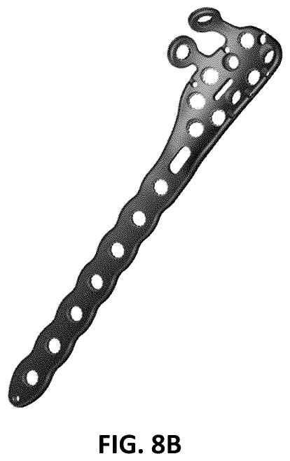

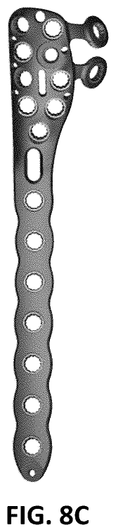

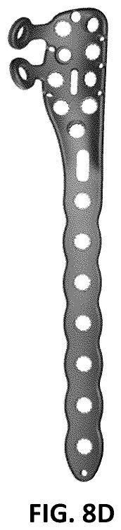

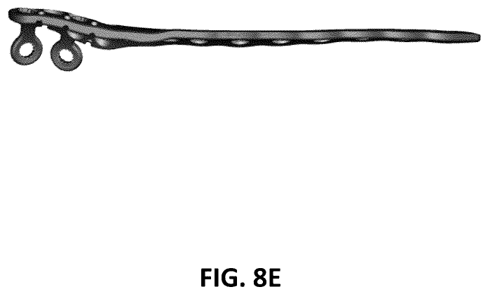

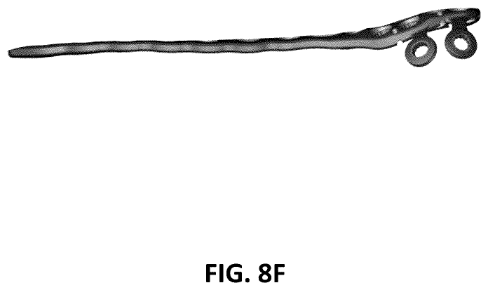

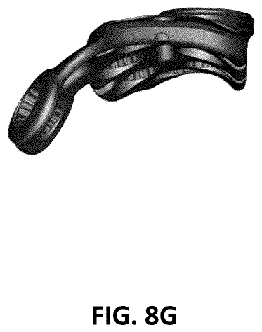

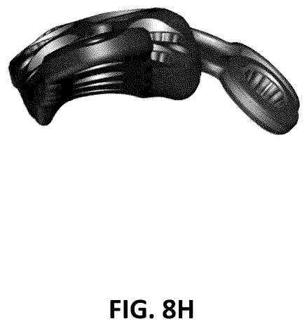

With reference to FIGS. 7 and 8A-H, a proximal humerus posterior plate 28 is shown. In addition to similar head, shafts and holes as in plate 22, plate 28 also includes two posterior extensions 30, 32. These extensions are easily bendable/deformable and include screw holes formed therethrough for the receipt of screws that can extend into the posterior aspect of the tibia. These permit screws to be placed into the greater tuberosity while counteracting the pull from the infraspinatus. These can be bent either by hand or utilizing tools such as bending sticks (not shown) to permit the best fit with patient anatomy. Like plate 22, plate 28 also includes two slots-one for receiving a k-wire and the other for receiving a bone screw. Like other plates included in system 10, plate 28 includes a plurality of other type holes, including k-wire holes and/or suture holes.

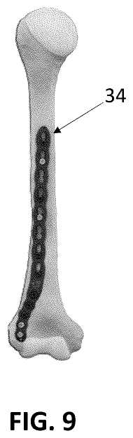

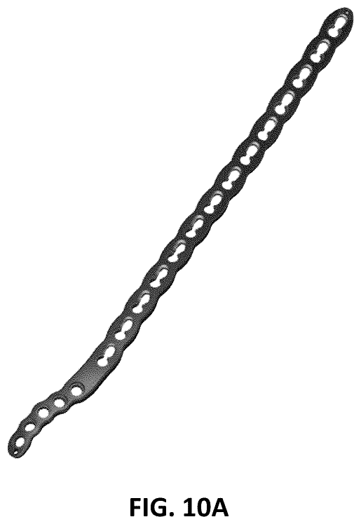

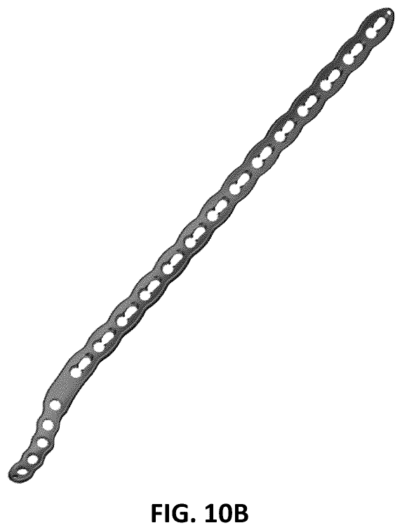

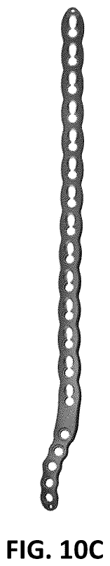

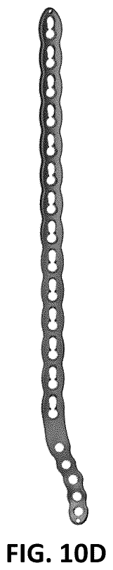

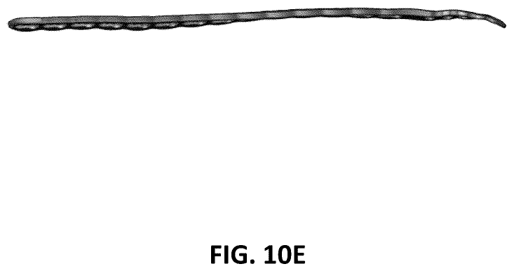

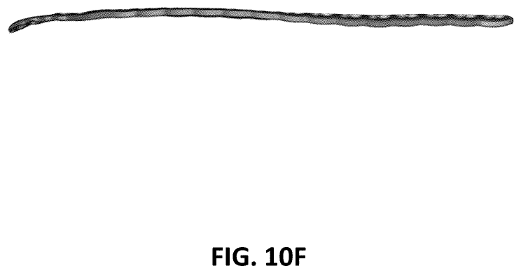

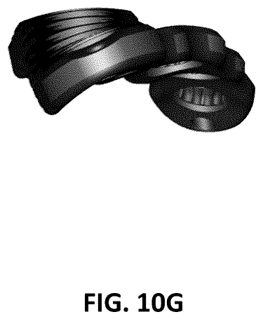

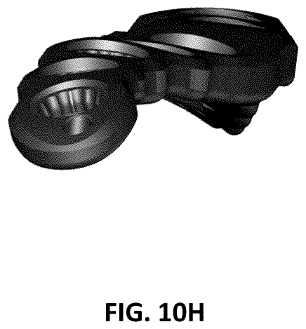

FIGS. 9 and 10A-H depict an extra articular distal humerus plate 34. As shown in FIG. 9, plate 34 is designed for placement on the distal humerus and includes a single row of plate holes extending along a curved and twisted plate body. Among the holes are hybrid locking/compression holes, which are discussed more fully below. Plate 34 includes an increased thickness that allows for single column plating.

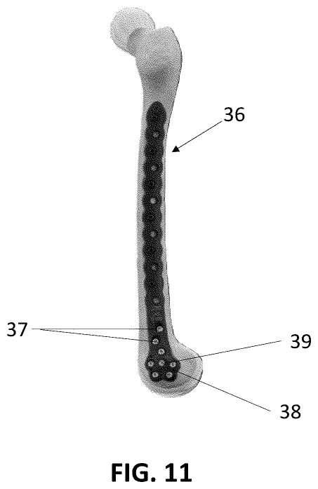

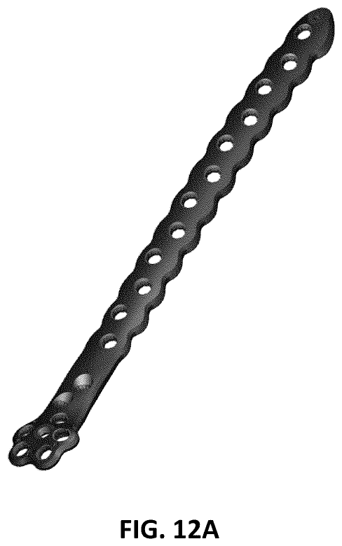

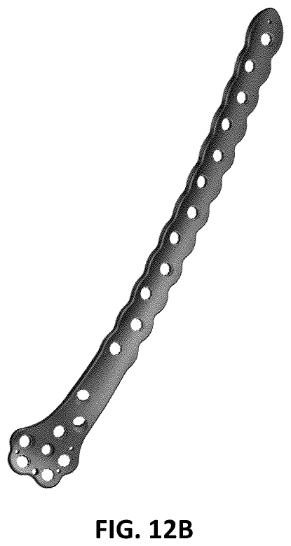

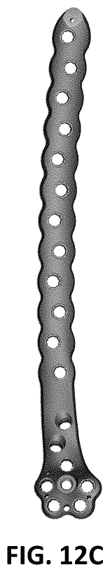

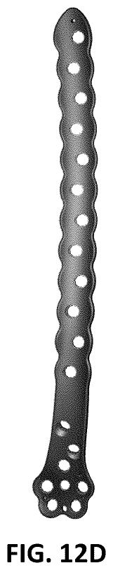

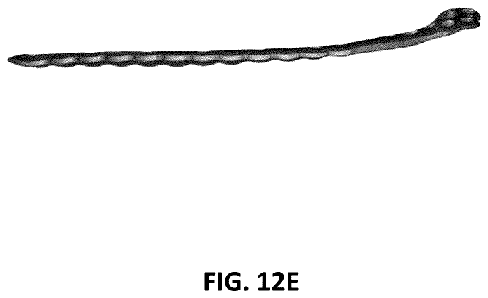

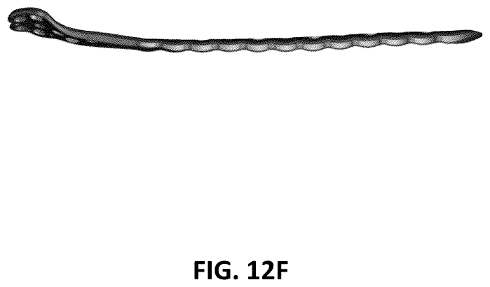

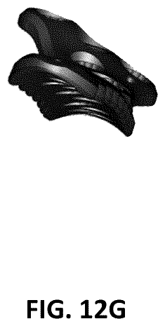

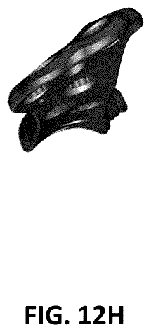

FIGS. 11 and 12A-H show a distal lateral femur plate 36. As shown in FIG. 11, plate 36 is designed for placement on the lateral aspect of the distal femur. Plate 36 includes holes similar to those discussed above in connection with other plates, as well as a head portion 38 including a plurality of holes and exhibiting an externally scalloped design. Head 38 is designed to permit the placement of screws into the distal portion of the femur in a manner that can reduce and fix complex fractures of the distal femur. Head 38 also includes a monoaxial hole 39 for receiving a monoaxial screw and/or threaded post, like is discussed above. The shaft of plate 36 includes stagger holes to accommodate screw placement around a nail or hip prosthesis and two kickstand holes 37 are included near head 38. These holes can accept screws that can extend to the medial femoral condyle.

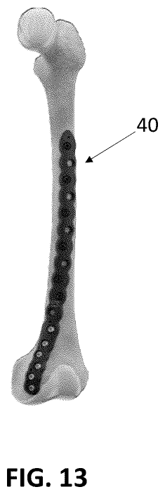

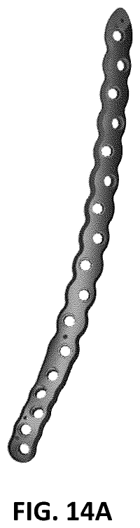

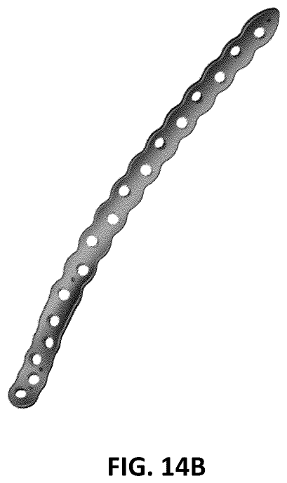

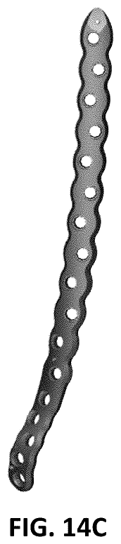

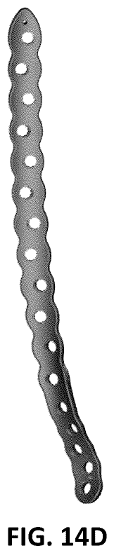

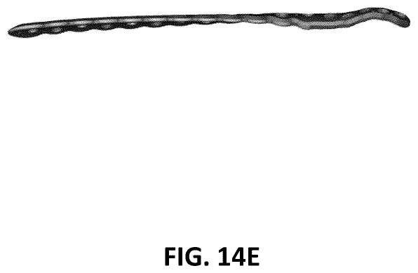

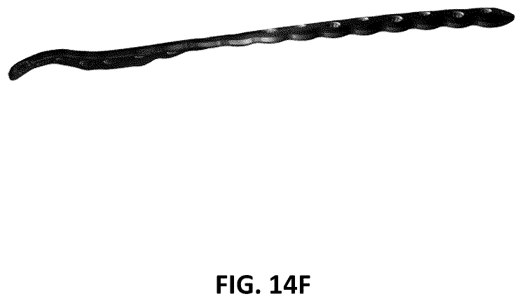

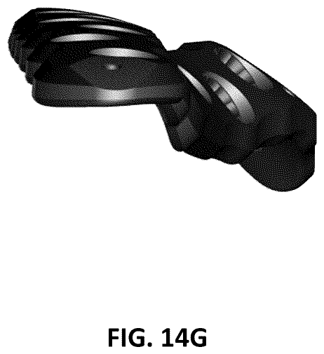

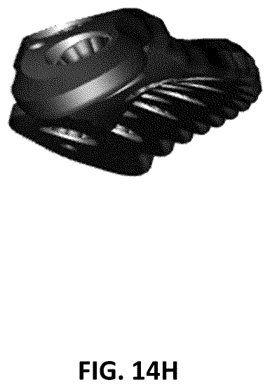

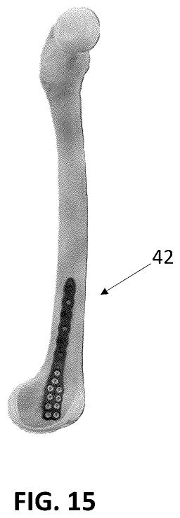

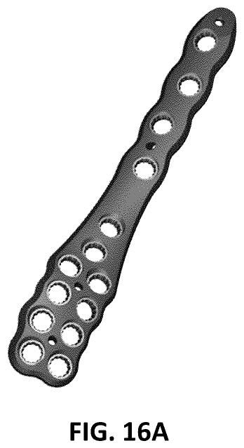

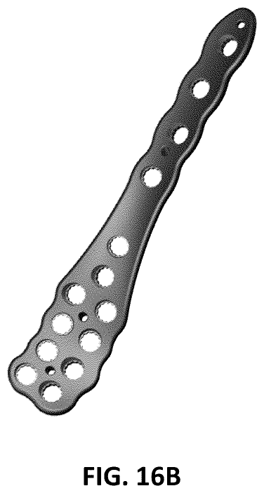

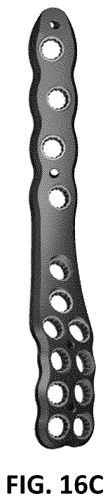

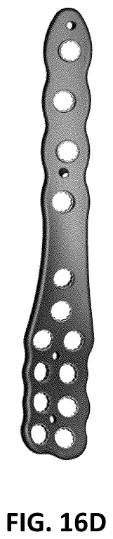

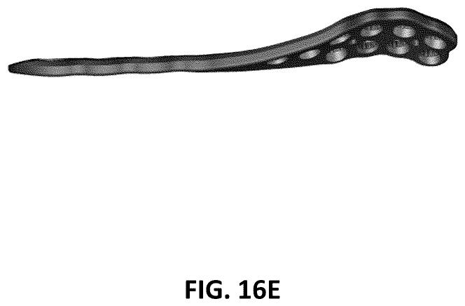

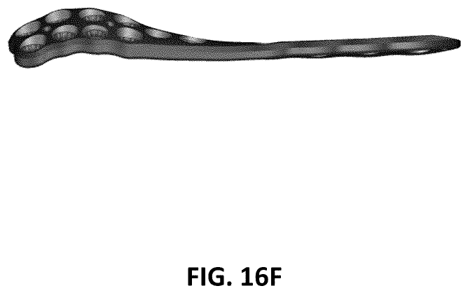

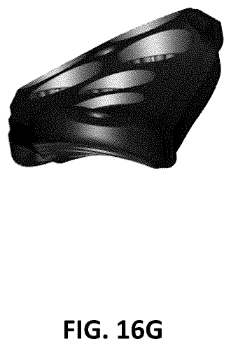

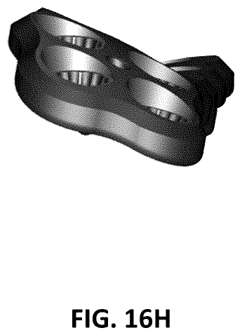

A first distal medial femur plate 40 is shown in FIGS. 13 and 14A-H and a second distal medial femur plate 42 is shown in FIGS. 15 and 16A-H. As shown, these plates are also designed for placement on a distal portion of the femur, albeit on a medial side. Like plate 34, plate 40 includes a single row of plate holes extending along a curved and twisted plate body. Plate 42 includes a more complex head than plate 40 with the ability to place more screws along different rows. Plate 40 is preferably utilized a medial based fixation for metadiaphyseal fracture. Plate 42 is preferably utilized for isolated medial condylar fractures. However, both plates are useful as a supplement to a lateral plate fixation when dual plate fixation is desired.

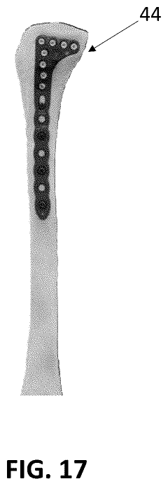

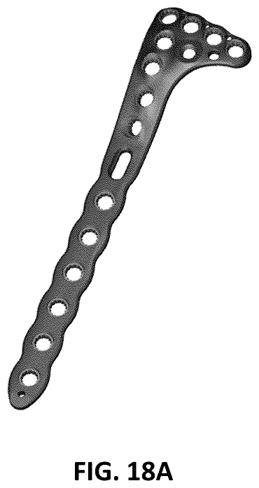

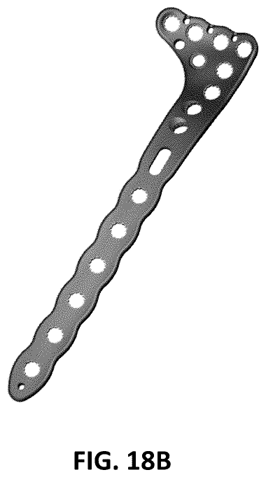

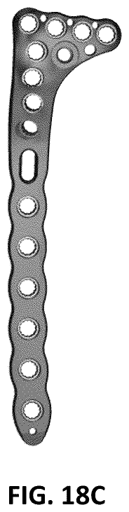

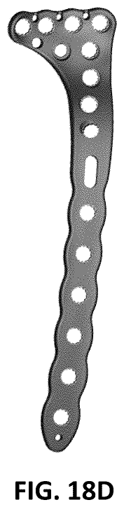

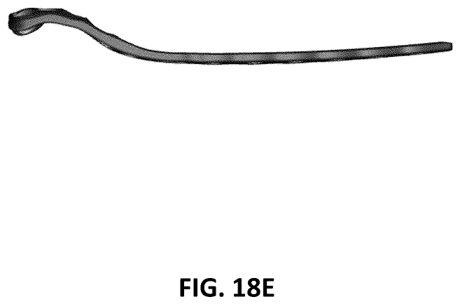

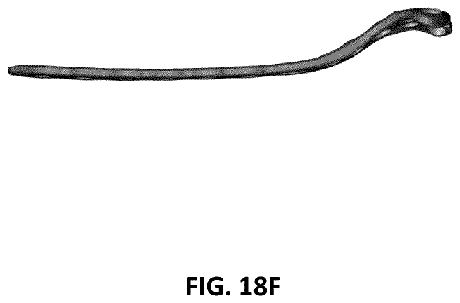

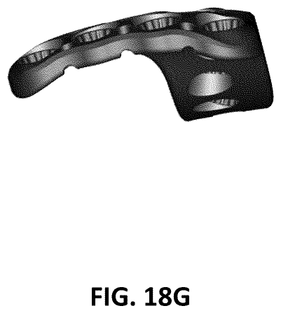

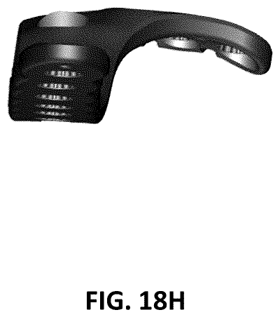

With reference to FIG. 17, a proximal lateral tibia plate 44 of system 10 is shown. Plate 44 is designed for placement on a lateral aspect of a proximal portion of the tibia. Plate 44 includes distinct head and shaft portions with the former having multiple rows of screw holes and the latter including a single row of screw holes. Like other plates, plate 44 includes a monoaxial hole, two kickstand holes and an oblong hole. The plate also includes a plurality of suture holes which permit the use of sutures and/or k-wires.

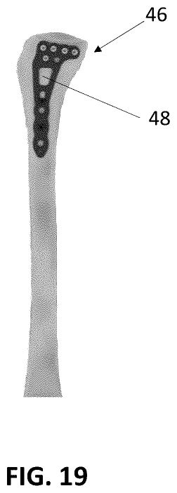

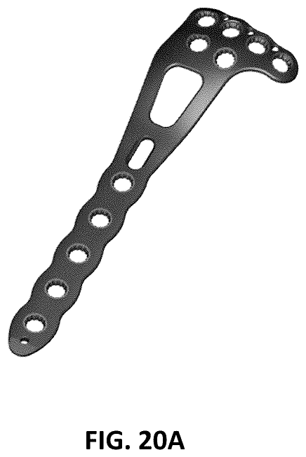

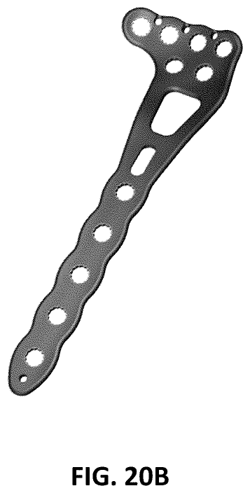

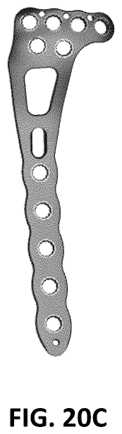

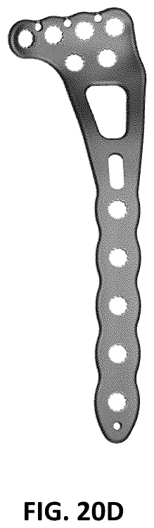

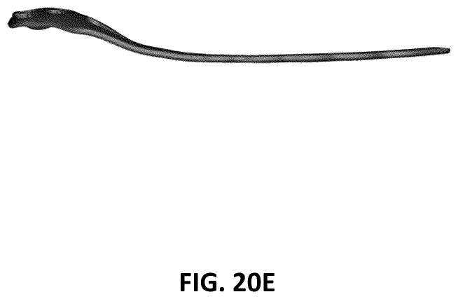

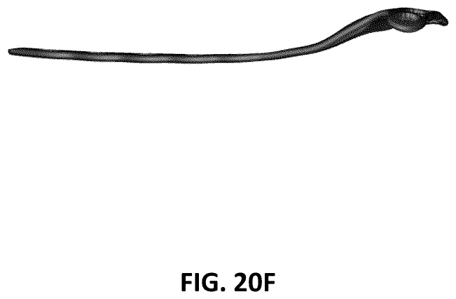

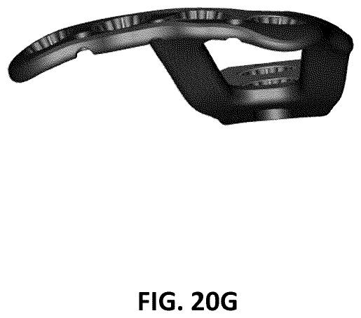

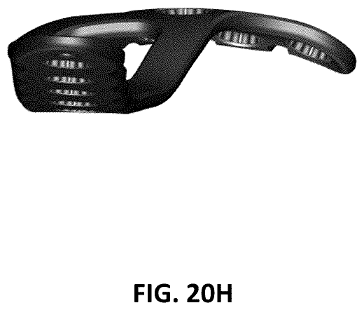

FIGS. 19 and 20A-H show a partial articular proximal tibia plate 46, which is also designed for placement on a lateral aspect of a proximal portion of the tibia. In addition to other holes similar to other plates of system 10, plate 46 includes a window 48 for optional tamping and grafting, which can be useful in certain procedures. This design is akin to that disclosed in U.S. patent application Ser. No. 18/232,511, now published as U.S. Patent Application Publication No. 2024/0050230, the disclosure of which is hereby incorporated by reference herein.

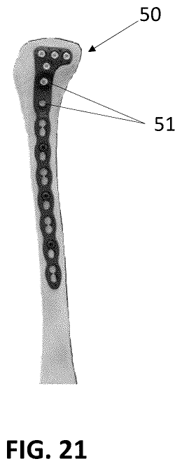

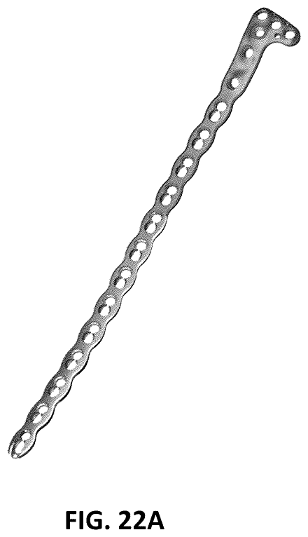

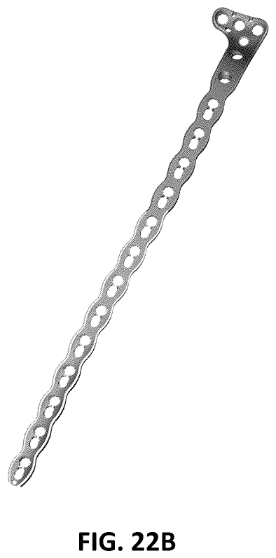

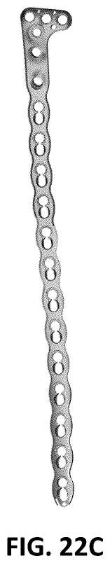

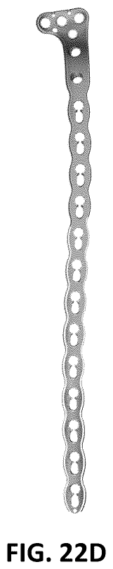

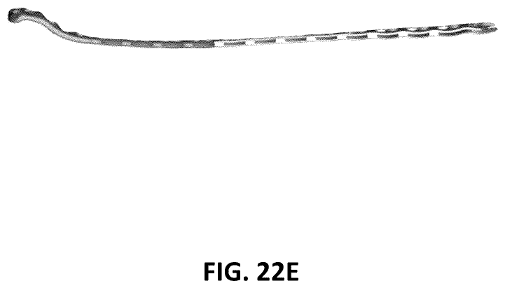

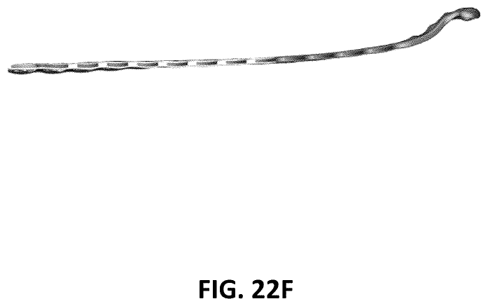

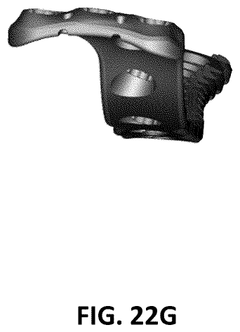

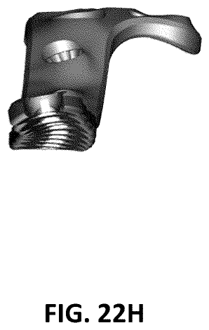

FIGS. 21 and 22A-H show an extra articular proximal tibia plate 50, which is also designed for placement on a lateral aspect of a proximal portion of the tibia. Plate 50 includes various holes similar to those of other plates of system 10, including some hybrid locking/compression holes. Plate 50 also includes two kickstand holes 51, which permit targeting of the medial tibial condyle.

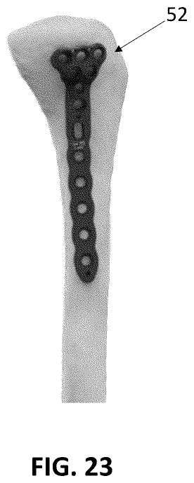

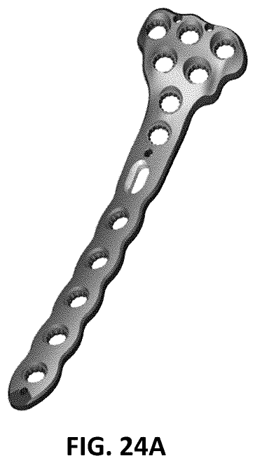

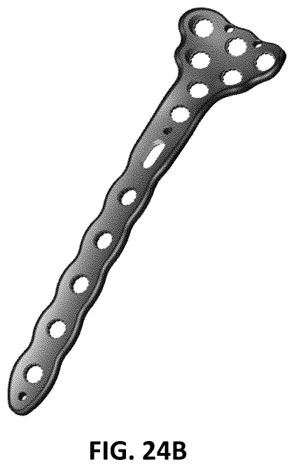

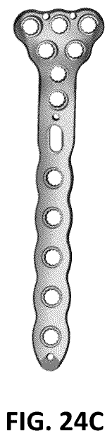

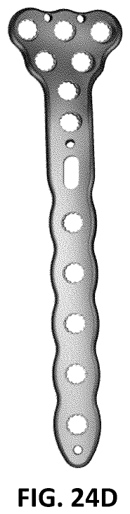

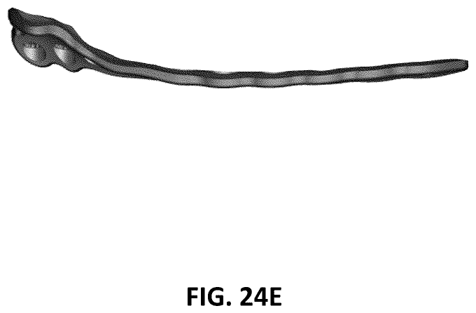

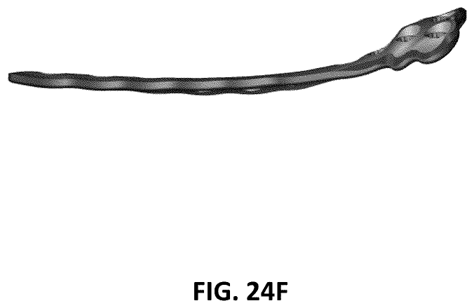

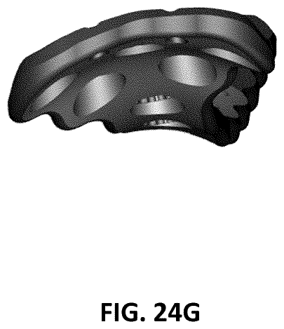

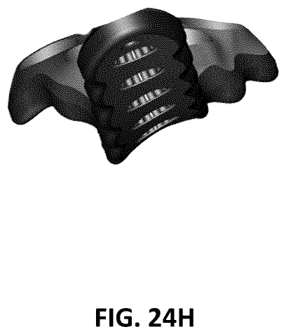

A proximal medial tibia plate 52 is shown in FIGS. 23 and 24A-H. Unlike the previous proximal tibia plates, plate 52 is designed for placement on a medial side of the tibia. In addition to a single row of holes included on a shaft portion, plate 52 includes a head portion with two rows of holes. Other features are similar to those of other plates discussed above.

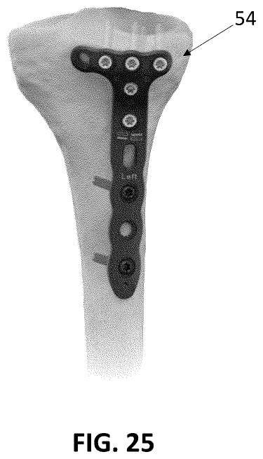

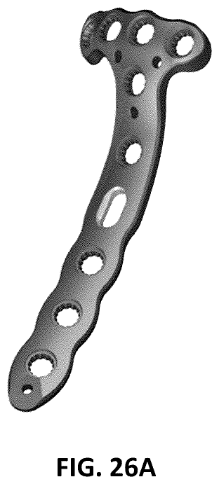

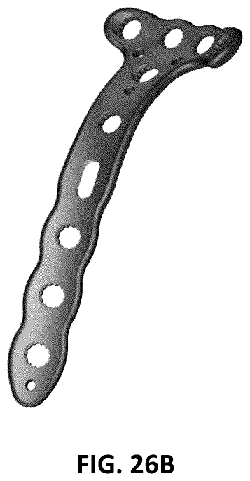

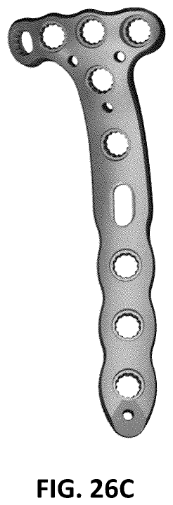

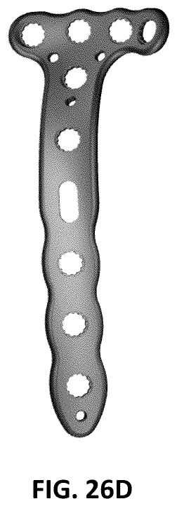

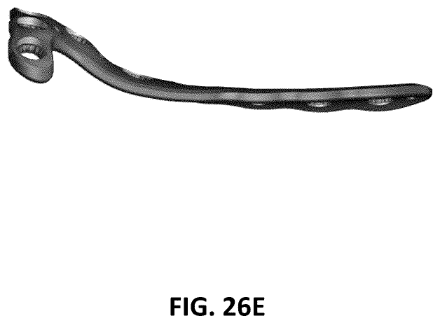

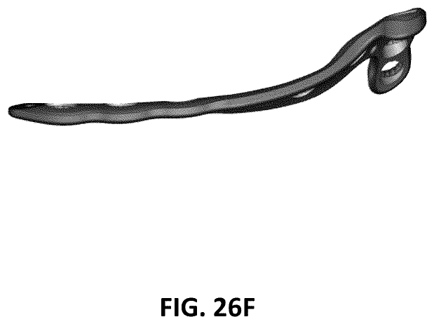

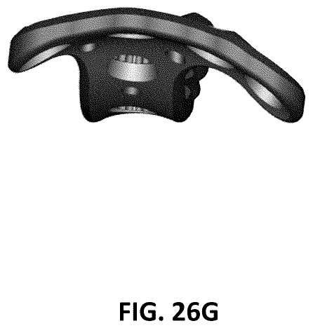

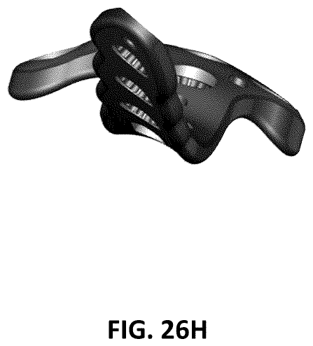

FIGS. 25 and 26A-H depict a proximal posteromedial tibia plate 54 included in system 10. While also designed for placement on a medial side like plate 52, plate 54 is designed to be placed more posteriorly. Plate 54 includes a shaft and a head, both with a single row of holes.

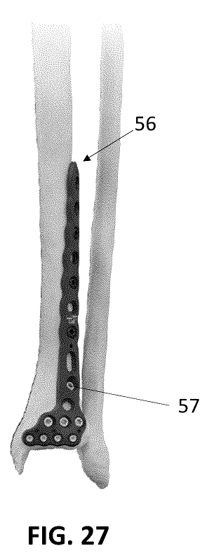

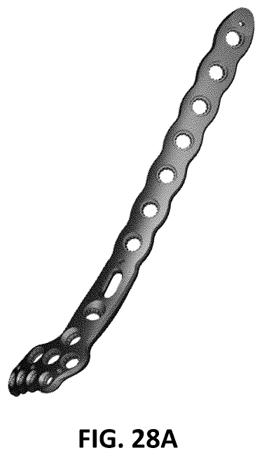

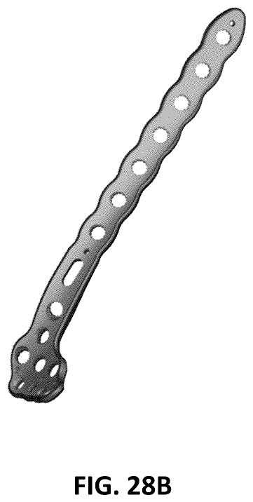

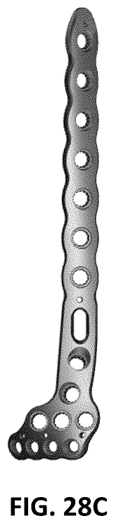

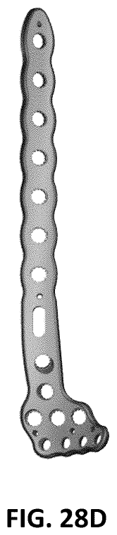

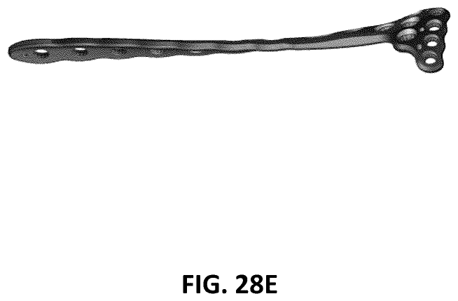

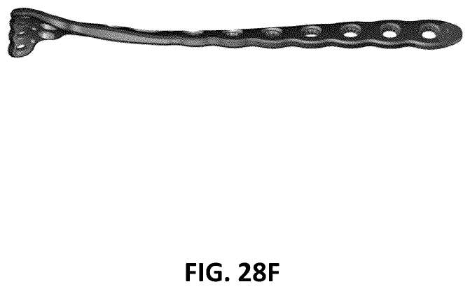

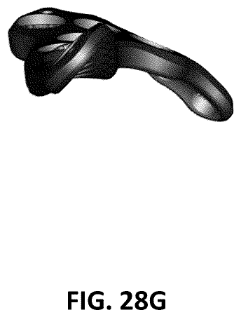

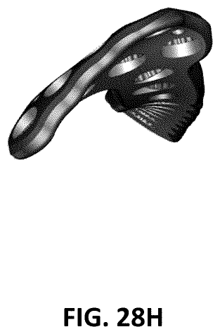

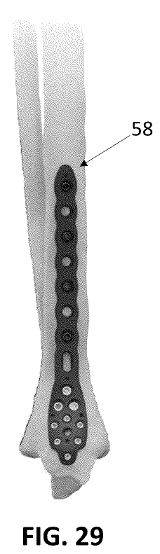

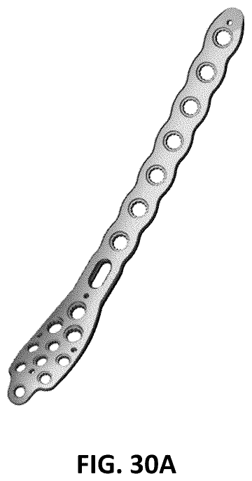

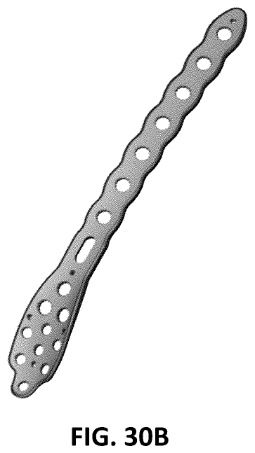

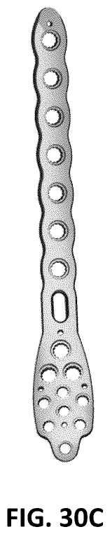

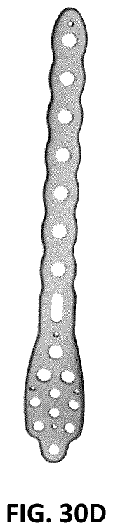

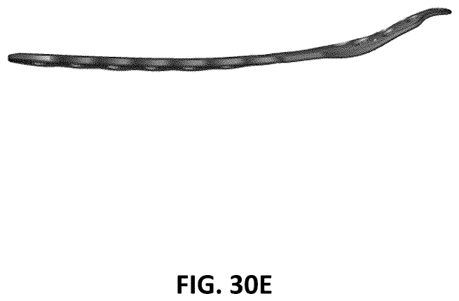

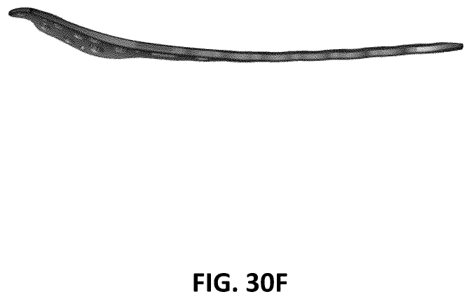

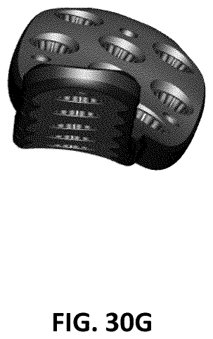

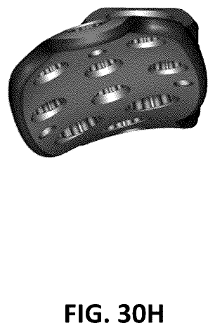

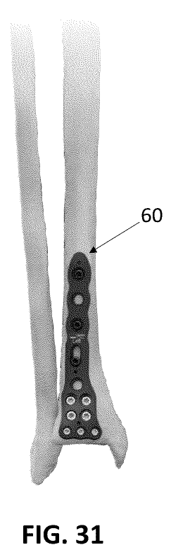

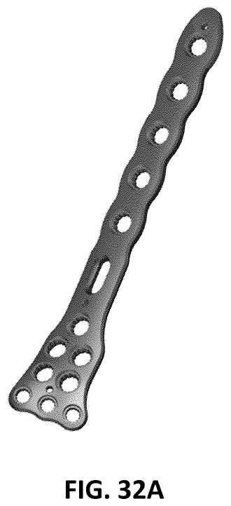

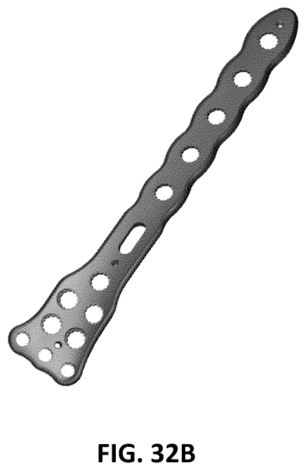

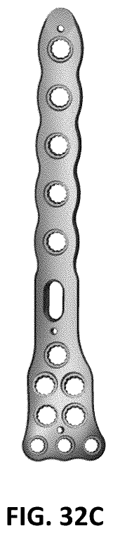

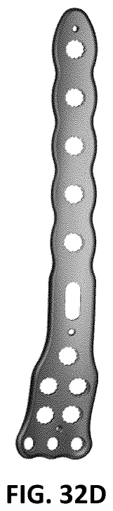

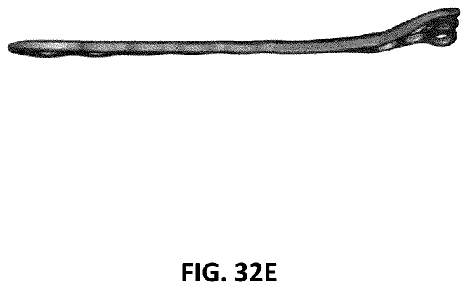

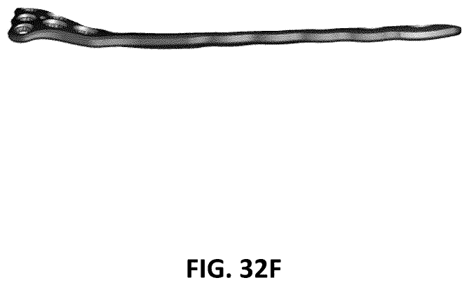

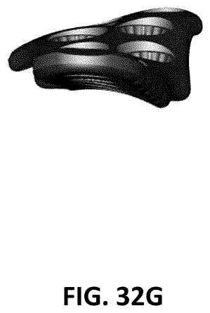

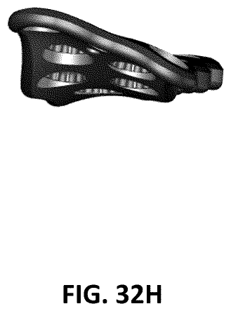

FIGS. 27 through 32H depict three different plates for placement on a distal tibia in an ankle surgery. For instance, a distal anterolateral tibia plate 56 is shown in FIGS. 27 and 28A-H, a distal medial tibia plate 58 is shown in FIGS. 29 and 30A-H and a distal posterior tibia plate 60 is shown in FIGS. 31 and 32A-H. These plates provide three separately designed plates for placement on different portions of the distal tibia. Plate 56 includes, among other holes, a kickstand hole 57 to target the medial malleolous. Plate 58 includes a head shape that is specifically designed to cooperate with the medial side of the distal tibia. Plate 60 similarly includes a specifically anatomically shaped head.

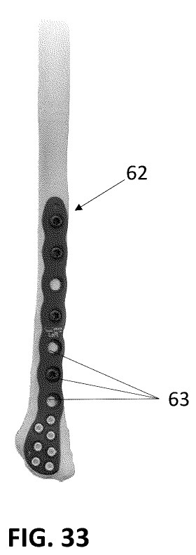

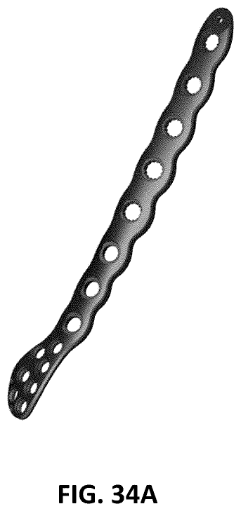

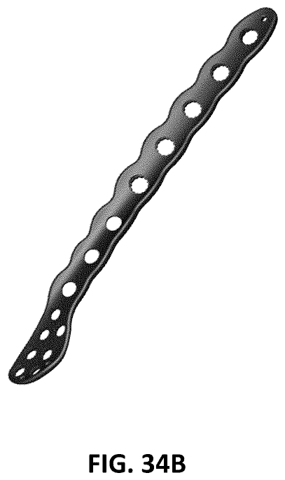

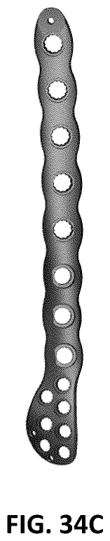

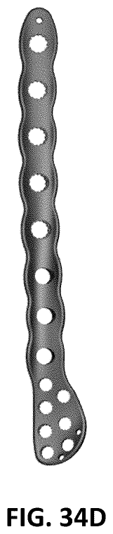

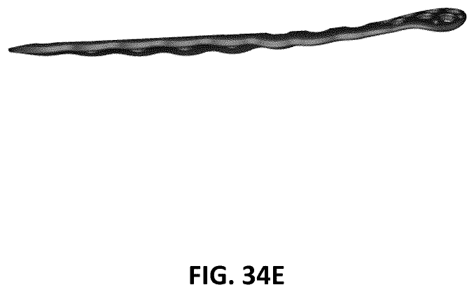

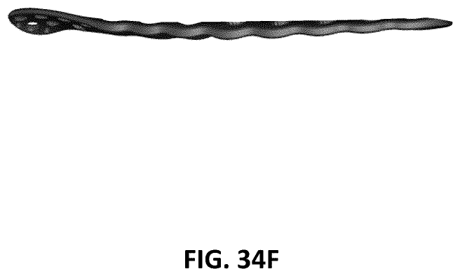

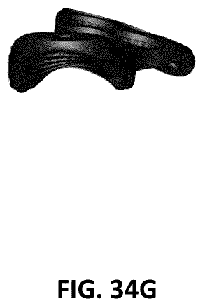

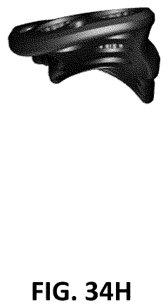

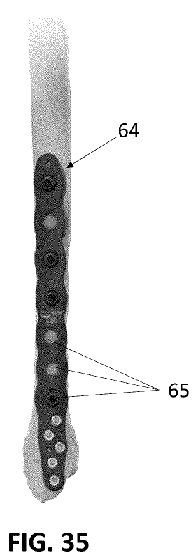

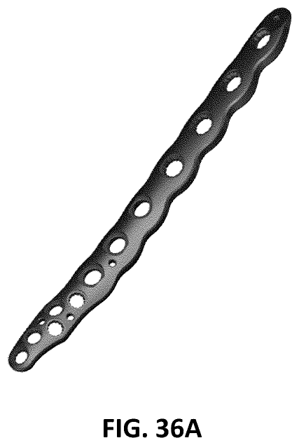

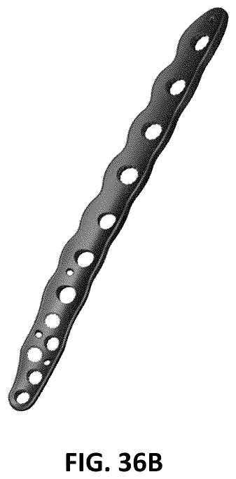

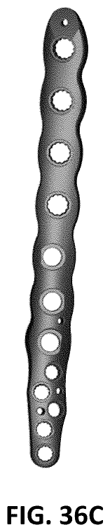

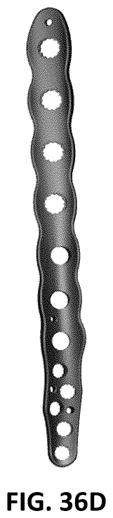

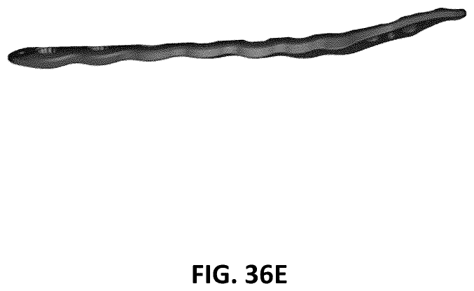

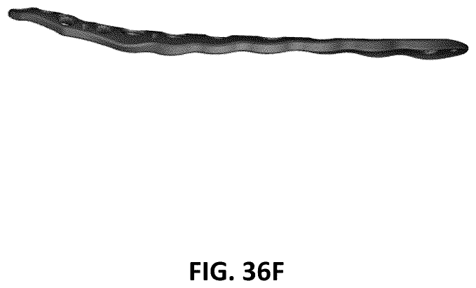

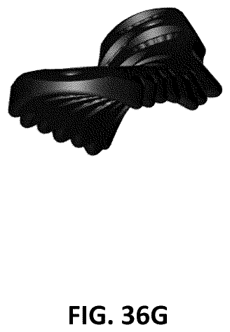

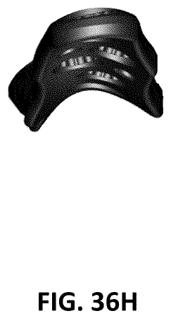

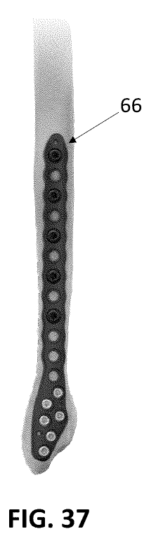

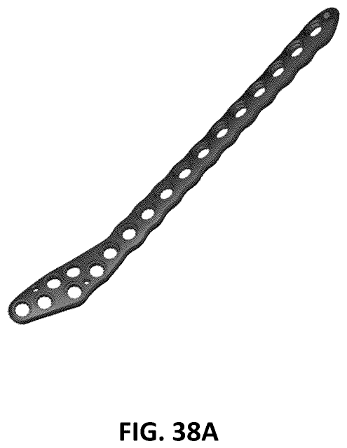

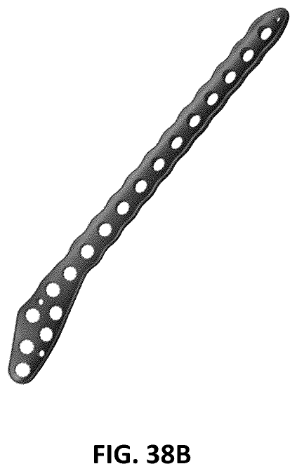

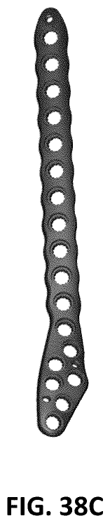

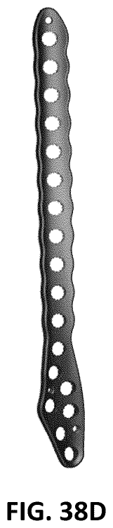

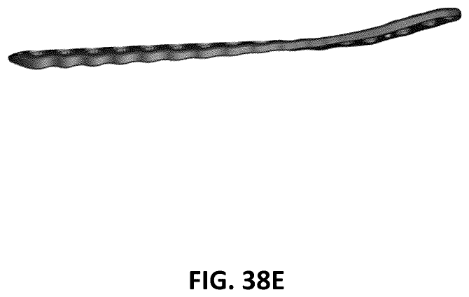

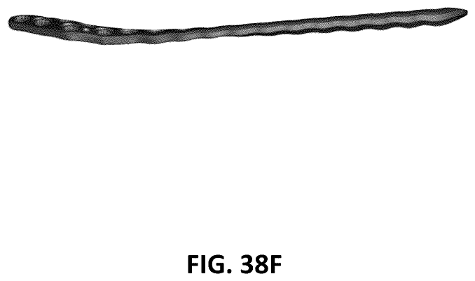

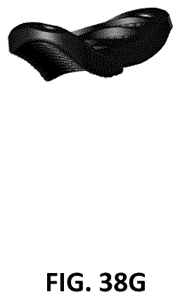

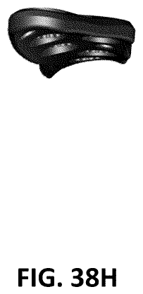

FIGS. 33 through 38H depict three different plates for placement on a distal fibula in an ankle surgery. For instance, a distal lateral fibula plate 62 is shown in FIGS. 33 and 34A-H, a distal posterolateral fibula plate 64 is shown in FIGS. 35 and 36A-H and a distal posterior fibula plate 66 is shown in FIGS. 37 and 38A-H. These plates provide three separately designed plates for placement on different portions of the distal fibula. Plates 62 and 64 include syndesmoditic holes-holes 63 and 65, respectively—that permit screws to pass through the plate, the fibula and into the tibia.

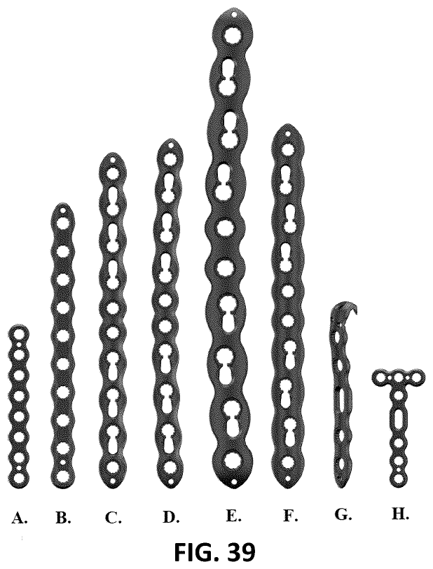

FIGS. 39A-G depict different utility plates included in system 10. For instance, FIGS. 39A-F depict relatively straight plates, while FIG. 39G depicts a hook plate and FIG. 39H depicts a t-shaped plate. These plates are useful in certain surgeries, either on their own or in conjunction with one or more of the anatomical plates discussed above. These utility plates include holes similar to those included in the plates discussed above.

FIG. 40 shows a hybrid locking/compression hole 70. As shown, hole 70 includes a variable angle locking hole portion 72 and a compression portion 74. While the former is essentially a semi-circular version of the variable angle locking holes discussed above, the latter exhibits more of a compression slot design. This design permits the insertion of any type of screw included in system 10, as well as the potential to reduce a fracture utilizing the same hole.

Certain of the plates of system 10 include multiple screw holes of different sizes. This means that different diameter screws can be utilized with the same plate. These hole sizes have been dictated by an analysis of the normal patient anatomy, which determined better fixation and healing based upon the differently sized screws. This is coupled with the general screw hole placement—also dictated by patient anatomy. For instance, distal anterolateral tibia plate 56 includes a head with differently sized screw holes oriented in rows. Of course, it is contemplated to vary the hole placement and/or screw sizes for each plate in other embodiments.

Various views of each bone plate are included in the accompanying drawings and are referred to above. It is to be understood that the visual ornamental characteristics embodied in each embodiment plate, in addition to any structural and/or functional features discussed above, form part of the inventions disclosed herein. Applicant reserves the right to file one or more design patents claiming priority to the present application under 35 U.S.C. § 120 or any other permissible provision under the law.

Although the inventions herein have been described with reference to particular embodiments, it is to be understood that these embodiments are merely illustrative of the principles and applications of the present inventions For example, features described in relation to one particular embodiment may be combined with features of other embodiments described herein. It is therefore to be understood that numerous modifications may be made to the illustrative embodiments and that other arrangements may be devised without departing from the spirit and scope of the present inventions as defined in the appended claims.

1. A bone plate system comprising:
a plurality of humerus plates;
a plurality of femur plates;
a plurality of tibia plates; and
a plurality of fibula plates.

2. The bone plate system of claim 1, wherein the humerus plates includes a proximal humerus plate, a proximal humerus posterior plate and an extra articular distal humerus plate.

3. The bone plate system of claim 1, wherein the femur plates include a distal lateral femur plate, a distal medial femur plate and a distal medial femur plate.

4. The bone plate system of claim 1, wherein the tibia plates include a proximal lateral tibial plate, a partial articular proximal tibia plate, an extra articular proximal tibial plate, a proximal medial tibial plate and a proximal posteromedial tibial plate.

5. The bone plate system of claim 4, wherein the tibia plates further include a distal anterolateral tibial plate, a distal medial tibia plate and a distal posterior tibia plate.

6. The bone plate system of claim 1, wherein the fibula plates include a distal lateral fibula plate, a distal posterolateral fibula plate and a distal posterior fibula plate.

7. The bone plate system of claim 1, further comprising a plurality of utility plates.

8. The bone plate system of claim 7, wherein the utility plates include straight plates, t-shaped plates and hook plates.

9. The bone plate system of claim 1, further comprising a tray of tooling for use with small fragment plates and screws.

10. The bone plate system of claim 1, further comprising a tray of tooling for use with large fragment plates and screws.

11. The bone plate system of claim 1, further comprising a screw rack including a plurality of differently sized screws.

12. The bone plate system of claim 11, wherein the screw rack includes a screw measuring scale.

13. The bone plate system of claim 12, wherein the screw rack includes a plurality of washer slots.

14. The bone plate system of claim 13, wherein the screws are arranged in discrete locations based upon size.

15. The bone plate system of claim 14, wherein the screw rack includes a removable cover.
